(19) United States
(12) Patent Application Publication
(10) Pub. No.: US 2009/0072866 A1
(43) Pub. Date: Mar. 19, 2009

(54) Method and system for controlling amplified signals reflecting physiological characteristics

(76) Inventor: Neil Edward Walker, Tampa, FL (US)

Correspondence Address: Ralph C. Francis;Francis Law Group, 1942 Embarcadero, Oakland, CA, 94606, US

(21) Appl. No.: 11/901,985

(22) Filed: Sep. 19, 2007

(51) Int. Cl. A61B 5/0428

(52) U.S. Cl. 327/62

(57) ABSTRACT

A method for controlling amplified signals in a medical diagnostic device having an amplifier system that is adapted to amplify input signals having a baseline and filter means for filtering the amplified signals, comprising the detection of the presence of an amplified signal having an amplitude that is above an upper voltage threshold or below a lower voltage threshold, and freezing the baseline of the amplified signal upon the detection thereof.

### FIELD OF THE PRESENT INVENTION

The present invention relates to the field of signal processing. More specifically, the invention relates to a method and system for controlling amplified signals in instruments employed in medical diagnoses and monitoring of physiological functions.

### BACKGROUND OF THE INVENTION

Physiological monitoring systems and apparatus that are adapted to acquire signals reflecting physiological characteristics are well known in the art. The noted systems include, for example, ECG and pulse oximetry systems.

The signals acquired by the noted physiological monitoring systems and apparatus are, however, composite signals, comprising a desired signal portion that directly reflects the physiological process that is being monitored and an undesirable signal portion, e.g., high-amplitude and interference signals. The undesirable signal portions or components often originate from both AC and DC sources.

Standard ECG systems, which typically employ amplifiers, are often subjected to high-amplitude or “overload” signals. The overload signals, can include, for example, defibrillation discharges, pacemaker pulses, noise transients and patient movements. Each of the noted overload signals can substantially shift the DC baseline of a normal low-level ECG signal. The overload signals can also create significant signal distortion, including amplifier clipping and saturation, wherein the normal signal is completely suppressed.

Further, since AC coupled amplifiers are employed for virtually all routine diagnostic and monitoring applications involving the measurement of electrical potentials associated with the human heart, the time constraints associated with the AC coupling capacitors in the signal processing amplifier must be relatively long to accurately reproduce the electrical signals, i.e. electrical potentials. Indeed, it is well known that time constraints that yield a low frequency 3 dB point in the range of approximately 0.5-0.05 Hz are commonly employed in ECG systems.

It is also well known that the use of long time constants can result in an undesirable amplifier response, such as baseline shift and other deleterious effects, in response to overload signals. The noted amplifier overload response can adversely affect a normal ECG signal and, hence, severely limit its clinical usefulness. The overload response can, and, in many instances, will cause a complete loss of the normal signal for several seconds.

Amplifier overload responses can also cause malfunctions in additional processing circuitry, such as heart rate meters, alarm sensing circuits, signal display scopes and hard copy recorders.

Unfortunately, in actual practice, ECG amplifiers are quite frequently driven into overload conditions. The signals that produce amplifier overload are typically classified into two broad groups; short, transient type signals, which can arise from a pacemaker pulse or radio frequency interference, and longer, extended type signals, which can result from electrode recovery following a defibrillator discharge or electrode movement. Whichever the type, it is evident that the disturbance of the charge on the AC coupling capacitor of the amplifier from its nominal value by the overload signal is the primary factor that results in an undesired residual amplifier response after the overload signal passes.

Various conventional methods and systems have been employed to abate overload signals. The noted methods and systems are typically designed to address either the short, transient type signals or the longer, extended type signals.

Illustrative are the slew rate detector circuits disclosed in U.S. Pat. Nos. 4,181,135, 4,149,527 and 5,762,068 and the “bootstrap” circuit disclosed in U.S. Pat. No. 4,319,197.

Slew rate detector circuits, such as the circuits disclosed in the U.S. patents referenced above, are typically employed to address short, transient type signal overloads. The slew rate circuits are designed to limit the amount of charge disturbance on the AC coupling capacitor by controlling the maximum rate of charge (increase or decrease) in the capacitor and, hence, suppress or cancel the overload signals that are present in a normal ECG signal.

While slew rate limiting is an effective means for suppressing transient disturbances, it has various inherent disadvantages that restrict its utility. For example, slew rate limiting requires a compromise between the high frequency signal handling ability of the amplifier and the amount of transient suppression desired. This represents a definite disadvantage, since reproduction of the higher frequency components is desirable for certain clinically encountered heart potentials, such as large amplitude, rapidly changing signals associated with pediatric patients, neonatal patients, and certain invasive measurements on adult patients.

Further, slew rate processing can, and in many instances will, adversely affect the signal path frequency response, e.g., introduce other forms of distortion into the ECG signal, and leave remnants of the overload signals, e.g., noise pulses, embedded in the ECG signal. The embedded remnants can cause a misinterpretation of important ECG characteristics, such as narrow QRS complexes.

Slew rate processing also cannot effectively suppress baseline movement without also suppressing or distorting important information that the overload pulse may convey; e.g. pacemaker timing characteristics. Indeed, when slew rate limiting is applied to the degree necessary to suppress to a negligible level any baseline disturbance of a normal ECG signal, a pacemaker “spike” is so suppressed that it is difficult to determine the temporal relationship between the “spike” and the ECG signal. The ability to “see” the pacemaker “spike” without undue disturbance of the normal ECG signal or the heart rate counting circuitry is particularly important for diagnostic procedures and research studies such as pacemaker-cardiac capture mechanisms or certain high rate atrial pacing techniques.

The “bootstrap” circuit disclosed in U.S. Pat. No. 4,319,197 is similarly, primarily designed to address short, transient type signal overloads. The “bootstrap” circuit provides a linear replica of the portion of an input signal that exceeds a predetermined constant (deemed an overload signal), and applies the replica to the coupling capacitor in such a manner as to oppose the change that would normally occur in the capacitor charge as a result of such overload.

Since the predetermined constant is preferably selected to be equal to the nominal full scale range of the system, an otherwise overloading signal appears to the coupling capacitor as not more than a nominal full scale signal regardless of amplitude, wave shape or duration. As a result, the time constant associated with the coupling capacitor can be sufficiently large to ensure proper signal reproduction without fear of deleterious amplifier response to overloading input signals.

The “bootstrap” circuit is thus similarly an effective means for suppressing transient disturbances. There are, however, several drawbacks and disadvantages associated with the noted circuit and means. A major drawback is that the circuit is very complex. A further drawback is that the circuit employs a gain adjustment that is based on a system determinative scaling factor. An additional drawback is that important information that may be present in the overload signal, such as pacemaker timing, is eliminated from the signal stream.

It would therefore be desirable to provide an improved method and system for abating the deleterious effects of high-amplitude and/or overload signals in signals reflecting physiological characteristics that can be effectively employed in medical diagnostic and monitoring apparatus and systems, while simultaneously preserving the fidelity of both the desired and overload signals to aid in advanced diagnostics

It is therefore an object of the present invention to provide an improved method and system for abating the deleterious effects of high-amplitude and/or overload signals in signals reflecting physiological characteristics, which can be effectively employed in medical diagnostic and monitoring apparatus and systems.

It is another object of the invention to provide a method and system for abating the deleterious effects of high-amplitude and/or overload signals in signals reflecting physiological characteristics, which substantially suppress baseline displacement that is otherwise caused by short, transient type overload signals, as well as longer, extended type overload signals.

A further object of the invention is to provide a method and system for abating the deleterious effects of high-amplitude and/or overload signals in signals reflecting physiological characteristics, which do not degrade the fidelity of the desired signal and the overload signal.

It is yet another object of the invention to provide a method and system for abating the deleterious effects of high-amplitude and/or overload signals that would otherwise degrade the DC stability, linearity and bandwidth of signals reflecting physiological characteristics, such as an ECG signal.

### SUMMARY OF THE INVENTION

In accordance with the above objects and those that will be mentioned and will become apparent below, in one embodiment of the invention, there is provided a method for controlling amplified signals in a medical diagnostic device, the device including an amplifier system that is adapted to receive and amplify input signals having a baseline, and filter means for filtering the amplified signals, the method comprising the steps of (i) detecting the presence of an overload input signal, the overload input signal comprising an amplified signal having an amplitude that is above an upper voltage threshold or below a lower voltage threshold, and (ii) freezing the baseline of the overload signal upon the detection thereof.

In one embodiment of the invention, the method includes the step of providing a hold circuit that is adapted to perform the detection of the overload signal and the freezing of the overload signal baseline, the hold circuit being in communication with the amplifier system and filter means, the hold circuit including first detection means for detecting the presence of the overload input signal and abatement means for abating transmission of the overload input signal through the filter means upon the detection thereof.

In one embodiment, the upper voltage threshold is in the range of approximately 200-300 mV±10%.

In one embodiment, the lower voltage threshold is in the range of approximately −200 to −300 mV±10%.

In another embodiment of the invention, the method includes the step of providing a reset circuit, the reset circuit being in communication with the filter means and adapted to reset the filter means when the overload signal time period exceeds an overload signal duration threshold.

In one embodiment, the overload signal duration threshold is in the range of approximately 50 ms to 5 sec.

In one embodiment, the overload signal duration threshold is in the range of approximately 450 ms to 550 ms.

In one embodiment of the invention, the reset circuit includes second detection means for detecting when the overload signal time period exceeds the overload signal duration threshold, and reset means for resetting the filter means when the overload signal time period exceeds the overload signal duration threshold.

In accordance with another embodiment of the invention, there is provided a control system for controlling amplified signals in a medical diagnostic device, the device including an amplifier system that is adapted to receive and amplify input signals, and filter means for filtering the amplified signals, the control system comprising a hold circuit that is in communication with the amplifier system and filter means, the hold circuit including (i) first detection means for detecting the presence of an overload signal, the overload signal comprising an amplified signal having an amplitude that is above an upper voltage threshold or below a lower voltage threshold, and (ii) abatement means for abating transmission of the overload signal through the filter means upon detection thereof.

In accordance with another embodiment of the invention, there is provided a control system for controlling amplified signals in a medical diagnostic device, the device including an amplifier system that is adapted to receive and amplify input signals, and filter means for filtering the amplified signals, the control system comprising (i) a hold circuit that is in communication with the amplifier system and filter means, the hold circuit including first detection means for detecting the presence of an overload signal, the overload signal comprising an amplified signal having an amplitude that is above an upper voltage threshold or below a lower voltage threshold, and abatement means for abating transmission of the overload signal through the filter means upon detection thereof, and (ii) a reset circuit that is adapted to reset the filter means when the overload signal amplitude is above the upper voltage threshold or below the lower voltage threshold for an overload signal time period that exceeds an overload signal duration threshold.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of an “r-wave” portion of an electrocardiogram (ECG) waveform or signal and the related plethysmographic waveform;

FIG. 2 is a schematic illustration of the r-wave portion of the ECG signal containing a large overload signal;

FIG. 3 is a schematic illustration of a conventional ECG device with an ECG amplifier arrangement employing one embodiment of the method and system of the invention;

FIG. 4 is a schematic illustration of one embodiment of a window comparator and associated circuitry, according to the invention;

FIG. 5 is a schematic illustration of a conventional ECG device with an ECG amplifier arrangement employing another embodiment of the method and system of the invention;

FIG. 6 is a schematic illustration of one embodiment of a delay comparator and associated circuitry, according to the invention;

FIG. 7 is a schematic illustration of a signal showing a baseline shift that results from an overload pulse;

FIG. 8 is a further schematic illustration of the response shown in FIG. 7 with the time scale compressed to illustrate the lengthy recovery time; and

FIG. 9 is a schematic illustration of a signal with an overload pulse, wherein the baseline DC shift has been prevented, but with the desired and overload signal fidelity otherwise preserved, according to the invention.

### DETAILED DESCRIPTION OF THE INVENTION

Before describing the present invention in detail, it is to be understood that this invention is not limited to particularly exemplified methods, systems or circuitry as such may, of course, vary. Thus, although a number of methods and systems similar or equivalent to those described herein can be used in the practice of the present invention, the preferred methods and systems are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise.

### DEFINITIONS

The term “signal”, as used herein, is meant to mean and include an analog electrical waveform or a digital representation thereof, which is collected from a biological or physiological sensor.

The term “signal baseline”, as used herein, is meant to mean and include the average value, e.g., voltage, of a signal or plurality of signals that is established at the output of an amplifier system.

The term “desired signal component”, as used herein, is meant to mean and include the portion of a signal that directly corresponds to the biological or physiological function being monitored.

The tern “undesirable signal component”, as used herein, is meant to mean and include any portion of a signal that does not correspond to the biological or physiological function being monitored. As such, the term includes, without limitation, noise, interference, and other variables that hinder the measurement of the biological or physiological function.

The terms “overload signals” and “high-amplitude signals” are used interchangeable herein and are meant to mean and include, without limitation, any signal having an undesirable extraneous component that is coupled onto a desired signal and/or signal component, such as a normal input ECG signal.

The term “freezing”, as used herein in connection with a signal, e.g., input signal, is meant to mean and include, without limitation, substantially maintaining the normal baseline of a signal that is exhibited in the absence of an overload condition.

The term “amplifier system”, as used herein, is meant to mean and include, without limitation, a system that is adapted to increase the voltage, current or power of a signal.

The terms “filter” and “filter system”, as used herein, are meant to mean and include a circuit or system that alters the voltage, current or power of a signal according to the signal's frequency and/or phase.

The terms “patient” and “subject”, as used herein, are meant to mean and include humans and animals.

The present invention substantially reduces or eliminates the disadvantages and drawbacks associated with prior art methods and systems for controlling amplified signals. As discussed in detail below, the methods and systems of the invention effectively negate the deleterious effects of amplified or overload signals in a simple and direct manner by (i) identifying an overload signal and (ii) freezing the baseline thereof. In one embodiment of the invention, the baseline is frozen, i.e. maintained at a pre-overload level, by increasing the time constant of a conventional high pass filter to near-infinity.

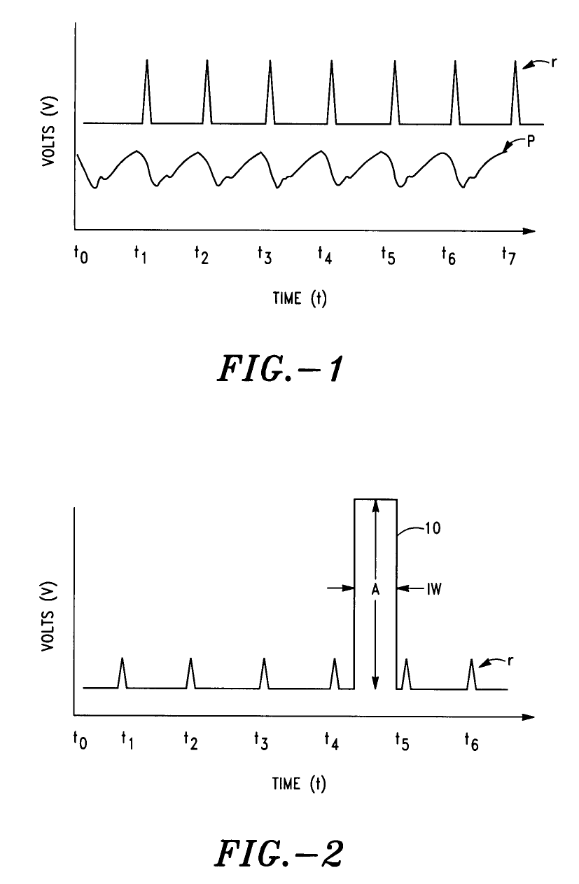

Referring first to FIG. 1, there is shown a schematic illustration of an “r-wave” portion of an electrocardiogram (ECG) waveform or signal (designated “r”) and the related plethysmographic waveform (designated “p”). As will be appreciated by one having ordinary skill in the art, the ECG waveform comprises a complex waveform having several components that correspond to electrical heart activity. The QRS component relates to ventricular heart contraction.

The r-wave portion of the QRS component is typically the steepest wave therein, having the largest amplitude and slope, and can be used for indicating the onset of cardiovascular activity. The arterial blood pulse flows mechanically and its appearance in any part of the body typically follows the R wave of the electrical heart activity by a determinable period of time that remains essentially constant for a stable patient. See, e.g., Goodlin et al., Systolic Time Intervals in the Fetus and Neonate, Obstetrics and Gynecology, Vol. 39, No. 2, (February 1972) and U.S. Pat. No. 3,734,086.

Referring now to FIG. 2, there is shown a schematic illustration of the r-wave portion of the ECG signal containing a large overload signal 10 that has an amplitude “A” and a width (or time period) of “TW”. As indicated above and discussed in detail below, the deleterious effects of such overload signals are readily abated by the methods and systems of the invention.

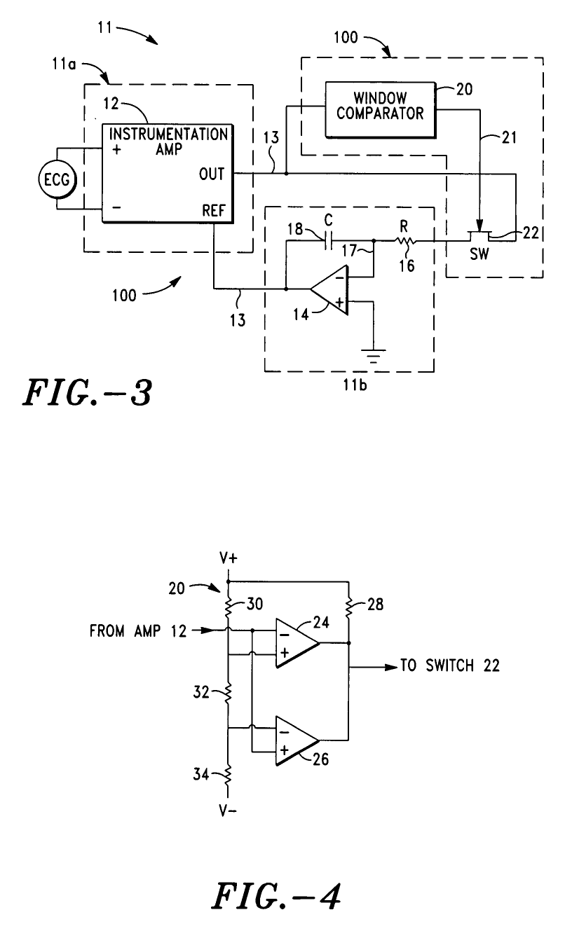

Referring now to FIGS. 3-9, the methods and systems of the invention will be described in detail. It should, however, be understood that the systems, instruments and circuits employed herein to describe the methods and systems of the invention are not meant to limit the scope of the inventions in any manner. Indeed, as will be appreciated by one having ordinary skill in the art, the methods and systems of the invention can be readily employed (or incorporated) in a multitude of electronic instrumentation and systems, including medical diagnostic and monitoring devices and systems.

Referring now to FIG. 3, there is shown a conventional medical diagnostic device or system 11 employing one embodiment of the methods and systems of the invention (hereinafter referred to generally as a hold circuit or system “100”). The medical diagnostic device 11, in this instance, is adapted to monitor ECG signals that are generated by a subject or patient.

As illustrated in FIG. 3, the medical device or system 11 includes an amplifier system (shown in phantom and designated “11a”) that is adapted to receive and amplify input signals, a filter or filter system (shown in phantom and designated “11b”) that is adapted to filter the amplified signals, the hold circuit or system 100 referenced above (also shown in phantom) and a system circuit or circuitry 13, which facilitates communication by and between systems 11a, 11b and 100.

In the illustrated embodiment, the amplifier system 11a includes an instrumentation amplifier 12 and the filter system 11b includes circuit 17, resistor 16, capacitor 18 and op amp 14, which is configured as a low pass filter, wherein the filter system 11b is driving the reference input of the amplifier 12. The noted configuration results in an overall high pass response for the amplifier 12, with the time constant set by the resistor 16 and capacitor 18.

Referring back to FIG. 3, the hold circuit 100 of the invention includes a window comparator 20, analog switch 22 and associated circuitry 21. According to the invention, the window comparator 20 is adapted to detect the presence of signals at the amplifier output that have an amplitude above or below the predetermined amplitude range or voltage threshold of the system 100.

As is well known in the art, a normal ECG signal range is approximately 0.5-5 mvpk. The normal ECG signal is then typically amplified to obtain the maximum linear output from the instrumentation amplifier (i.e. amplifier 12). A factor of 10 or higher is typical, but this can vary considerably depending on design constraints (DC or AC coupling, input voltage, supply voltage, output stage limits, etc.). AC coupling is typically used to eliminate the high biopotential DC offset (e.g. ±300 mv) and related drift, which, in turn, allows the gain of the instrumentation amplifier to be set higher for an improved signal-noise ratio. For the embodiment(s) discussed herein, common conditions are an instrumentation amplifier gain of 25, V+=+5V±5% and V−=−5V±5%,

According to the invention, during overload intervals, i.e. presence of an overload signal, the window comparator 20 turns off analog switch 22, which effectively increases the filter time constant to near-infinity, i.e. the filter is held at its last DC state. This increase in time constant has three valuable attributes: (i) extended low frequency operation is not interrupted or degraded, (ii) the DC baseline shift that would otherwise be introduced by the overload signal is prevented, and (iii) the fidelity of the desired signal and overload signal are preserved, which allows a downstream processor to extract valuable diagnostic information from the overload signal (e.g., precise pacemaker timing), and/or allow the overload signal to be precisely subtracted from the desired signal.

Since the window comparator 20 effectively senses any out-of-range signal, it is therefore effective at detecting pacemaker, interference spikes, electrode displacement step changes, or any other type of high-amplitude noise.

Referring now to FIG. 4, there is shown one embodiment of a window comparator 20 of the invention. As illustrated in FIG. 4, the window comparator 20 includes two comparators 24, 26. The comparators 24, 26 preferably include open-drain outputs that are coupled to a pull-up resistor 28, and positive and negative trip points that are set by bias network, i.e. resistors 30, 32, 34.

In the noted embodiment, the pull-up resistor preferably comprises a 100K 5% 1/16 W resistor. Preferably, resistors 30, 32 and 34 comprise a 100K, 10K and 100K resistor; each 1% 1/16 W.

According to the invention, under normal operation, the amplifier 12 output signal amplitude does not exceed the pre-set trip points and the dual comparator output is high, which maintains analog switch 22 in an “on” state. If the amplifier 12 output exceeds the positive trip point, i.e., upper voltage threshold, or is lower than the negative trip point, i.e. lower voltage threshold, the dual comparator output transitions to a negative voltage (i.e. negative voltage output signal), which turns off analog switch 22 for the duration of the overload pulse.

As will be appreciated by one having ordinary skill in the art, the upper and lower voltage thresholds are subject to variation based on instrument settings, e.g., amplifier gain set to a different value.

In one embodiment of the invention, the upper voltage threshold is preferably in the range of 200-300 mV±10%, more preferably, the upper voltage threshold is in the range of 100-200 mV±10%, even more preferably, the upper voltage threshold is approximately 150 mV±1%, and the lower voltage threshold is preferably in the range of −200-−300 mV±10%, more preferably, the lower voltage threshold is in the range of −100-−200 mV±10%, even more preferably, the lower voltage threshold is approximately −150 mV±1%.

In accordance with one embodiment of the invention, there is thus provided a control system for controlling amplified signals in a medical diagnostic device, the device including an amplifier system that is adapted to receive and amplify input signals, and filter means for filtering the amplified signals, the control system comprising a hold circuit that is in communication with the amplifier system and filter means, the hold circuit including (i) detection means for detecting the presence of an overload signal, the overload signal comprising an amplified signal having an amplitude that is above an upper voltage threshold or below a lower voltage threshold, and (ii) abatement means for abating transmission of the overload signal through the filter means upon detection thereof.

In a preferred embodiment of the invention, the hold circuit substantially negates a shift in the overload signal baseline upon detection thereof.

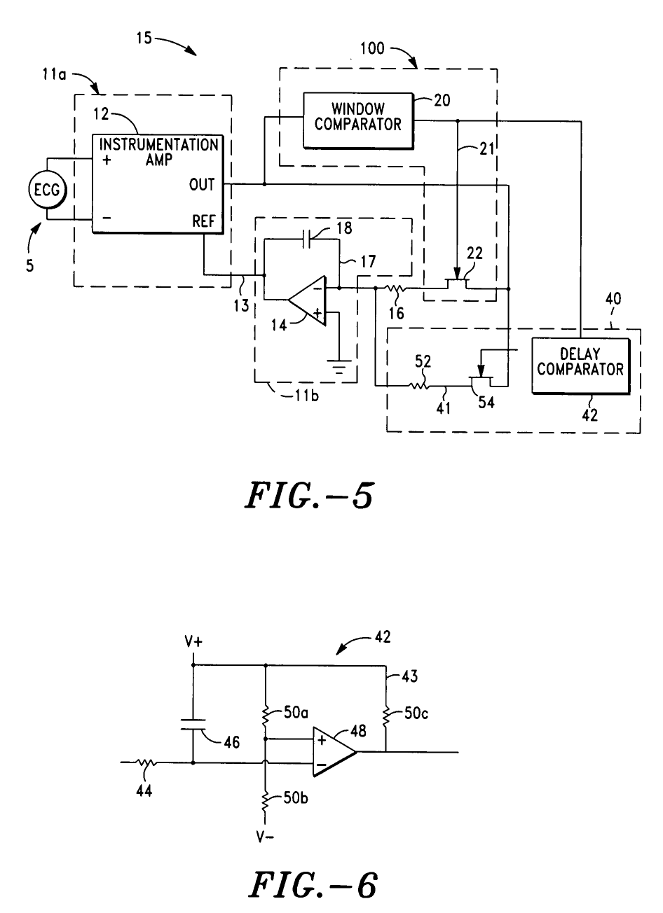

Referring now to FIG. 5, there is shown another embodiment of the methods and systems of the invention. As illustrated in FIG. 5, in this embodiment the system (designated generally “15”) similarly includes system 11 shown in FIG. 3 and discussed above. The system 15 does, however, include an optional “reset” circuit or system (shown in phantom and designated “40”), which, according to the invention, is adapted to reset the filter 11B in the event of an excessively long overload condition. In a preferred embodiment of the invention, the reset circuit 40 includes a delay comparator 42 and associated circuitry 41.

Referring now to FIG. 6, there is shown one embodiment of a delay comparator 42 and associated circuitry 43. As illustrated in FIG. 6, in one embodiment of the invention, the delay comparator 42, in simple form, comprises a resistor 44-capacitor 46 delay that is coupled to a single comparator 48. The delay comparator circuit 43 also includes pull-up resistor 50c and bias resistors 50a, 50b.

According to the invention, if the output of the window comparator 20 shown in FIG. 5 persists for more than the resistor 44×capacitor 46 time constant, then comparator 48 activates analog switch 54. When analog switch 54 is activated, resistor 52 is coupled to the op amp 14 (or filter circuit 17), which allows the filter system 11b to rapidly reset according to the time constant of resistor 52×capacitor 18. Preferably, the value of resistor 52 is selected to be the lowest practical design value to accomplish the quickest reset.

In one embodiment, the resistor 44×capacitor 46 time constant is preferably in the range of approximately 50 ms to 5 sec. In another embodiment, the resistor 44×capacitor 46 time constant is in the range of approximately 450-550 ms.

Thus, in accordance with another embodiment of the invention, there is provided a control system for controlling amplified signals in a medical diagnostic device, the device including an amplifier system that is adapted to receive and amplify input signals, and filter means for filtering the amplified signals, the control system comprising (i) a hold circuit that is in communication with the amplifier system and filter means, the hold circuit including first detection means for detecting the presence of an overload signal, the overload signal comprising an amplified signal having an amplitude that is above an upper voltage threshold or below a lower voltage threshold, and abatement means for abating transmission of the overload signal through the filter means upon detection thereof, and (ii) a reset circuit that is adapted to reset the filter means when the overload signal amplitude is above the upper voltage threshold or below the lower voltage threshold for an overload signal time period that exceeds an overload signal duration threshold.

In a preferred embodiment of the invention, the reset circuit includes second detection means for detecting when the overload signal time period exceeds the overload signal duration threshold, and reset means for resetting the filter means when the overload signal time period exceeds the overload signal duration threshold.

The effectiveness of the methods and systems of the invention and, hence, systems 11 and 15 above, can be closely approximated by using the following formulas to compute the baseline distortion and recovery time that would occur without the methods and systems of the invention:

Vbos=A×G×tW/(R×C) Eq. 1

tREC=5×R×C Eq. 2
where:
Vbos=baseline offset voltage step that would occur due to a rectangular overload pulse of amplitude A and width tW;
tREC=baseline recovery time (to within 1% of original baseline) following end of overload pulse;
A=input overload pulse amplitude, i.e. peak volts;
G=gain of instrumentation amplifier;
tW=overload pulse width (seconds);
R=filter resistor (ohms); and
C=filter capacitor (farads).

By way of illustration, when a system having a filter resistor of 5 megohms, a filter capacitor of 1 uF, and an instrumentation amplifier gain of 25 experiences an overload pulse of 100 mv over a duration of 0.5 seconds, the baseline shift would be a step of 250 mv and the recovery time would be approximately 25 seconds.

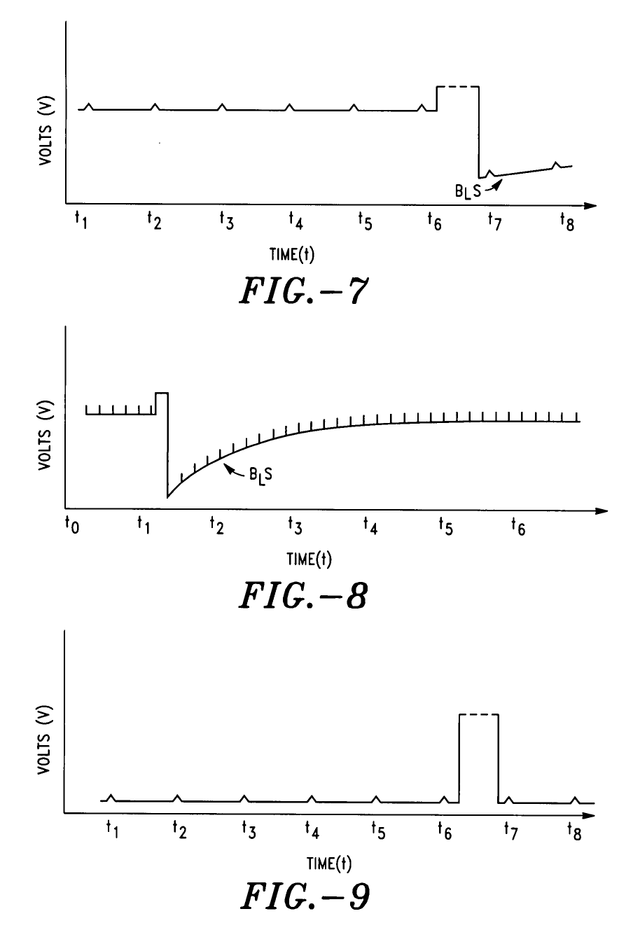

For a typical ECG input of 1 mvpk, the normal ECG signal at the amplifier (e.g., amplifier 12 above) output would be 25 mvpk. Therefore, referenced to the amplifier output, the overload pulse causes a baseline step shift of ten times the normal ECG peak amplitude. This output response is graphically illustrated in FIG. 7, wherein the baseline step shift is generally designated “B_{L}S”.

Referring now to FIG. 8, there is shown the same response with the time scale compressed to illustrate the lengthy recovery time.

Referring to FIG. 9, there is shown a graphical illustration of the output response with a hold circuit of the invention active. As illustrated in FIG. 9, with a hold circuit active, there is negligible baseline shift.

The systems and methods described above thus substantially preserve the original normal signal content and its DC baseline during overload events. The systems and methods prevent overload-generated step changes in the baseline and the related lengthy recovery delays associated therewith, which can be difficult for downstream digital processing techniques to handle. Maintaining the DC baseline also prevents circuit railing or saturation during which the normal ECG signal can be completely lost.

Without departing from the spirit and scope of this invention, one having ordinary skill in the art can make various changes and modifications to the invention to adapt it to various usages and conditions. As such, these changes and modifications are properly, equitably, and intended to be, within the full range of equivalence of the following claims.

What is claimed is:

1. A control system for controlling amplified signals in an instrument system, comprising:
an amplifier system that is adapted to receive and amplify input signals, said amplified signals having a signal baseline;
filter means in communication with said amplifier system for filtering said amplified signals; and
a hold circuit in communication with said amplifier system and said filtering means, said hold circuit including detection means for detecting the presence of an overload signal, said overload signal comprising an amplified signal having an amplitude that is above an upper voltage threshold or below a lower voltage threshold, and abatement means for abating transmission of said overload signal through said filter means upon detection thereof.

2. (canceled)

3. The system of claim 1, wherein said hold circuit substantially negates a shift in said signal baseline upon detection of said overload signal.

4. (canceled)

5. The system of claim 1, wherein said detection means comprises a window comparator, said window comparator including a window detection circuit that is in communication with said filter means, said window comparator circuit being adapted to convert said overload signal to a negative voltage output signal in response to the input thereto of an overload signal amplitude that is greater than said upper voltage threshold or less than said lower voltage threshold.

6-7. (canceled)

8. The system of claim 1, wherein said abatement of said overload signal through said filter means extends for an overload signal time period, wherein said detection means detects a first overload signal amplitude that is above said upper voltage threshold or below said lower voltage threshold.

9. The system of claim 1, wherein said upper voltage threshold is in the range of approximately 200-300 mV +/−10%.

10-11. (canceled)

12. The system of claim 1, wherein said lower voltage threshold is in the range of approximately −200 to −300 mV +/−10%.

13-14. (canceled)

15. A control system for controlling amplified signals in a medical diagnostic device, the device including an amplifier system that is adapted to receive and amplify input signals, and filter means for filtering the amplified signals, the control system comprising:
a hold circuit in communication with the amplifier system and filtering means, said hold circuit including first detection means for detecting the presence of an overload signal, said overload signal comprising an amplified signal having an amplitude that is above an upper voltage threshold in the range of approximately 200-300 mV +/−10% or below a lower voltage threshold in the range of approximately −200 to −300 mV +/−10%, and abatement means for abating transmission of said overload signal through the filter means upon detection thereof.

16-18. (canceled)

19. The system of claim 2, wherein wherein said detection means comprises a window comparator, said window comparator including a window detection circuit that is in communication with the filter means and is adapted to convert said overload signal to a negative voltage output signal in response to the input thereto of overload signal amplitude that is greater than said upper voltage threshold or less than said lower voltage threshold.

20-21. (canceled)

22. The system of claim 15, wherein said abatement of said overload signal through the filter means extends for an overload signal time period, wherein said detection means detects a first overload signal amplitude that is above said upper voltage threshold or below said lower voltage threshold.

23-28. (canceled)

29. The system of claim 15, wherein the control system includes a reset circuit that is adapted to reset the filter means when said overload signal time period exceeds an overload signal duration threshold in the range of approximately 50 ms to 5 sec.

30-31. (canceled)

32. The system of claim 29 wherein said reset circuit includes second detection means for detecting when said overload signal time period exceeds said overload signal duration threshold, and reset means for resetting the filter means when said overload signal time period exceeds said overload signal duration threshold.

33-35. (canceled)

36. A control system for controlling amplified signals in a medical diagnostic device, the device including an amplifier system that is adapted to receive and amplify input signals, and filter means for filtering the amplified signals, the control system comprising:
a hold circuit in communication with the amplifier system and filtering means, said hold circuit including first detection means for detecting the presence of an overload signal, said overload signal comprising an amplified signal having an amplitude that is above an upper voltage threshold or below a lower voltage threshold, and abatement means for abating transmission of said overload signal through the filter means upon detection thereof; and
a reset circuit that is adapted to reset the filter means when said overload signal amplitude is above said upper voltage threshold or below said lower voltage threshold for an overload signal time period that exceeds an overload signal duration threshold.

37-51. (canceled)

52. The system of claim 36, wherein said reset circuit includes second detection means for detecting when said overload signal time period exceeds said overload signal duration threshold, and reset means for resetting the filter means when said overload signal time period exceeds said overload signal duration threshold.

53-55. (canceled)

56. A method for controlling amplified signals in a medical diagnostic device, the device including an amplifier system that is adapted to receive and amplify input signals having a baseline, and filter means for filtering the amplified signals, the method comprising the steps of:
detecting the presence of an overload input signal, said overload input signal comprising an amplified signal having an amplitude that is above an upper voltage threshold or below a lower voltage threshold; and
freezing the baseline of said overload signal upon said detection thereof.

57. The method of claim 56 including the step of providing a hold circuit that is adapted to perform said detection of said overload signal and said freezing of said overload signal baseline, said hold circuit being in communication with the amplifier system and filter means, said hold circuit including first detection means for detecting said presence of said overload input signal, and abatement means for abating transmission of said overload input signal through the filter means upon said detection thereof

58. The method of claim 57, wherein said transmission abatement of said overload input signal through the filter means extends for an overload signal time period, wherein said first detection means detects said presence of said overload input signal.

59. The method of claim 56, wherein said upper voltage threshold is in the range of approximately 200-300 mV +/−10%.

60. The method of claim 56, wherein said lower voltage threshold is in the range of approximately −200 to −300 mV +/−10%.

61. The method of claim 56, including the step of providing a reset circuit, said reset circuit being in communication with the filter means and adapted to reset the filter means when said overload signal time period exceeds an overload signal duration threshold in the range of approximately 50 ms to 5 sec.

62-63. (canceled)

64. The system of claim 61, wherein said reset circuit includes second detection means for detecting when said overload signal time period exceeds said overload signal duration threshold, and reset means for resetting the filter means when said overload signal time period exceeds said overload signal duration threshold.
